SEQUENCE LISTING (28 sequences)

SEQ ID NO: 1 - DNA, 70 bp, Artificial
  Truncated RNAI
acagtatttggtatctgcgctctgctgaagccagttaccttcggaaaaagagttggtagctcttgatccg

SEQ ID NO: 2 - DNA, 70 bp, Escherichia coli
  promoter (1)..(70)
gatcaaaggatcttcttgagatcctttttttctgcgcgtaatctgctgcttgcaaacaaaaaaaccaccg

SEQ ID NO: 3 - DNA, 70 bp, Artificial
  Mutated RNAII promoter sequence from PUC
gatcaaaggatcttcttgacatcctttttttctgcgtataatctgctgcttgcaaacaaaaaaaccaccg

SEQ ID NO: 4 - DNA, 56 bp, Escherichia coli
  promoter (1)..(56): RNAI promoter from PUC
cagagttcttgaagtggtggcctaactacggctacactagaaggacagtatttggt

SEQ ID NO: 5 - DNA, 56 bp, Artificial
  Mutated RNAI promoter from PUC
cagagttcaacttcacgtggcctaactacggcatgtgatcttgaacagtatttggt

SEQ ID NO: 6 - DNA, 111 bp, Escherichia coli
  tRNA (1)..(111): AlaU
ccgctcgagtaatacgactcactataggggctatagctcagctgggagagcgcctgctttgcacgcaggaggtctgcggttcgatcccgcatagctccaccaagatcttcc

SEQ ID NO: 7 - DNA, 111 bp, Artificial
  Mutated tRNA AlaU from E.coli
ccgctcgagtaatacgactcactatagcggctatagctcagctgggagagcgcctgctttgcacgcaggaggtctgcggttcgatcccgcatagctgcaccaagatcttcc

SEQ ID NO: 8 - DNA, 1927 bp, Artificial
  Construct containing RNAI sequence and lac promoter
gcggccgcgttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaaacatagaattcgatatcaagcttacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggctaagcataaatgtgagcggataacattgacattgtgagcggataacaagatactgagcacacagtatttggtatctgcgctctgctgaagccagttaccttcggaaaaagagttggtagctcttgatccggcaaacaaaccaccgttggtagcggtggtttttttgttctcgaggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaggtacc

SEQ ID NO: 9 - DNA, 39 bp, Artificial
  Primer
ctacggcatgtgatcttgaacagtatttggtatctgcgc

SEQ ID NO: 10 - DNA, 38 bp, Artificial
  Primer
ttaggccacgtgaagttgaactctgtagcaccgcctac

SEQ ID NO: 11 - DNA, 42 bp, Artificial
  Primer
gcgcggatcccgggctcgaggccactggagcacctcaaaaac

SEQ ID NO: 12 - DNA, 44 bp, Artificial
  Primer
gatgggatccagatcttctagagcatccatttattactcaaccg

SEQ ID NO: 13 - DNA, 53 bp, Artificial
  Primer
ccgctcgagtaatacgactcactatagcggctatagctcagctgggagagcgc

SEQ ID NO: 14 - DNA, 78 bp, Artificial
  Primer
ggaagatcttggtgcagctatgcgggatcgaaccgcagacctcctgcgtgcaaagcaggcgctctcccagctgagcta

SEQ ID NO: 15 - DNA, 58 bp, Artificial
  Primer
ccgctcgaggtagttaatacgactcactataggggctatagctcagctgggagagcgc

SEQ ID NO: 16 - DNA, 78 bp, Artificial
  Primer
ggaagatcttggtggagctatgcgggatcgaaccgcagacctcctgcgtgcaaagcaggcgctctcccagctgagcta

SEQ ID NO: 17 - DNA, 35 bp, Artificial
  Primer
gctgctgctagcatgatgtctagattagataaaag

SEQ ID NO: 18 - DNA, 36 bp, Artificial
  Primer
gctgctggatccttaagacccactttcacatttaag

SEQ ID NO: 19 - DNA, 59 bp, Artificial
  Primer
tcgattttctctatcactgatagggagtggtaaaataactctatcaatgattaaggagg

SEQ ID NO: 20 - DNA, 59 bp, Artificial
  Primer
tcgacctccttaatcattgatagagttattttaccactccctatcagtgatagagaaaa

SEQ ID NO: 21 - DNA, 39 bp, Artificial
  Primer
tagcggtggtttttttgttgagctcggtacccggggatc

SEQ ID NO: 22 - DNA, 40 bp, Artificial
  Primer
ggaacggtggtttgtttgcgcctagttctcgatggttgag

SEQ ID NO: 23 - DNA, 58 bp, Artificial
  Primer
cttccattgaccgaagtcgtctcgcgtctatggatgaaaggagaagaacttttcactg

SEQ ID NO: 24 - DNA, 61 bp, Artificial
  Primer
cctttttctcaaccatcgagaactaggccaattcatatgtatatctccttcttaaagttaa

SEQ ID NO: 25 - DNA, 88 bp, Artificial
  Primer
atgatggctaagcataaatgtgagcggataacattgacattgtgagcggataacaagatactgagcacacagtatttggtatctgcgc

SEQ ID NO: 26 - DNA, 30 bp, Artificial
  Primer
ccgctcgagaacaaaaaaaccaccgctacc

SEQ ID NO: 27 - DNA, 17 bp, Artificial
  Primer
gttgcgacggtggtacg

SEQ ID NO: 28 - DNA, 18 bp, Artificial
  Primer
tgaagaagttcgcgcgcg

(19) United States
(12) Patent Application Publication
(10) Pub. No.: US 2011/0269184 A1
(43) Pub. Date: Nov. 3, 2011

(54) METHOD FOR CONTROLLING PLASMID COPY NUMBER IN E.COLI

(75) Inventors: Reingard Grabherr, Pressbaum (AT); Ester Egger, Niederrussbach (AT); Stefan Gross, Niederrussbach (AT); Juergen Mairhofer, Vienna (AT)

(73) Assignee: Boehringer Ingelheim RCV GmbH & Co. KG, Vienna (AT)

(21) Appl. No.: 12/837,137

(22) Filed: Jul. 15, 2010

(30) Foreign Application Priority Data
Jul. 16, 2009 (EP) 09165666.0

(51) Int. Cl. C12P 21/00; C12N 15/63; C12N 15/64; C12N 1/21

(52) U.S. Cl. 435/69.1; 435/252.3; 435/320.1; 435/91.4

(57) ABSTRACT

Host vector system and methods for plasmid DNA and recombinant protein production. The system allows copy number control of a ColE1 plasmid in E. coli by an RNA molecule that is transcribed from the host's genome and that interacts with plasmid-transcribed RNAI or RNAII. The system can be extended to combine PCN control and antibiotic-free selection.

### BACKGROUND OF THE INVENTION

The use of plasmid DNA as gene transfer vehicle has become widespread in gene therapy, as well as for the production of recombinant proteins in various cell lines.

In gene therapy applications, a plasmid carrying a therapeutic gene of interest is introduced into patients; transient expression of the gene in the target cells leads to the desired therapeutic effect.

Recombinant plasmids carrying the therapeutic gene of interest are obtained by cultivation of bacteria. Large scale production by fermentation processes relies on optimized conditions in order to maximize yield and quality.

Recombinant protein production in E. coli also relies on plasmid propagation. The gene encoding the target protein is present on the plasmid, transcribed and translated by the host's synthesis machinery.

Plasmid replication puts a load on the host's metabolic machinery, which sometimes leads to hampered cell growth or loss of plasmid. It has been shown that during recombinant protein production the concentration of unloaded tRNAs increases, thereby interaction with replication regulatory RNAs occurs and plasmid copy number is deregulated, increases drastically and causes termination of the production process (Wrobel and Wegrzyn, 1998). Mutations within the origin of replication can prevent the interaction with unloaded tRNAs and avoid uncontrolled increase of plasmid copy number (Grabherr et al., 2002; WO 02/29067). The mechanism of replication and the plasmid copy number (PCN) of plasmids depend on the DNA sequence of the origin of replication. So far, in fermentation processes, PCN has been regulated exclusively by modifications of the plasmid or by fermentation conditions.

A large number of naturally occurring plasmids as well as many of the most commonly used cloning vehicles are ColE1-type plasmids. These plasmids replicate their DNA by using a common mechanism that involves synthesis of two RNA molecules, interaction of these molecules with each other on the one hand and with the template plasmid DNA on the other hand (Helinski, 1996; Kues and Stahl, 1989).

Representatives of ColE1-type plasmids are the naturally occurring ColE1 plasmids pMB1, p15A, pJHCMW1, as well as the commonly used and commercially available cloning vehicles such as pBR322 and related vectors, the pUC plasmids, the pET plasmids and the pBluescript vectors (e.g.: Bhagwat, 1981; Balbas, 1988; Bolivar, 1979; Vieira, 1982). For all these plasmids, ColE1 initiation of replication and regulation of replication have been extensively described (e.g.: Tomizawa, 1981, 1984, 1986, 1990a, 1990b; Chan, 1985; Eguchi, 1991a, 1991b; Cesareni, 1991). The ColE1 region contains two promoters for two RNAs that are involved in regulation of replication. Replication from a ColE1-type plasmid starts with the transcription of the pre-primer RNAII, 555 by upstream of the origin of replication, by the host's RNA polymerase. During elongation, RNAII folds into specific hairpin structures and, after polymerization of about 550 nucleotides, begins to form a hybrid with the template DNA. Subsequently, the RNAII pre-primer is cleaved by RNase H to form the active primer with a free 3′ OH terminus, which is accessible for DNA polymerase I (Lin-Chao and Cohen, 1991; Merlin and Polisky, 1995).

At the opposite side of the ColE1-type origin strand, RNAI, an antisense RNA of 108 nucleotides, complementary to the 5′ end of RNAII, is transcribed. Transcription of RNAI starts 445 by upstream from the replication origin and continues to approximately the starting point of RNAII transcription. RNAI inhibits primer formation and thus replication by binding to the elongating RNAII molecule before the RNA/DNA hybrid is formed.

The interaction of the RNAI and RNAII is a stepwise process, in which RNAI and RNAII form several stem loops. They initially interact by base-pairing between their complementary loops to form a so-called “kissing complex”. Subsequently, RNAI hybridizes along RNAII, and a stable duplex is formed. Formation of the kissing complex is crucial for inhibition of replication. As it is the rate limiting step, is has been closely investigated (Gregorian, 1995). Apart from RNAI/RNAII interaction, the rom/rop transcript of ColE1 contributes to plasmid copy number (PCN) control by increasing the rate complex formation between RNAII and

RNAI.

To increase copy number, the gene encoding rom/rop has been deleted on some derivatives of pBR322, for example on pUC19.

It has been an object of the invention to provide a host-vector system that allows for controlled regulation of the PCN in order to diminish the metabolic load during fermentation, in particular during the exponential phase. Such system should be applicable both for large scale production of pDNA and for the production of recombinant proteins, which both rely on the propagation of plasmids.

In order to minimize the metabolic load during exponential growth, it is desirable to keep PCN low until the late phase of fermentation. Therefore, in the case of DNA production, it is desirable to enhance PCN towards the end of the process.

The solution of the problem is based on modulating (enhancing or reducing) plasmid replication at a selected point of time, i.e. when the cell density has reached the desired level, whereby said modulation is accomplished from the host genome, i.e. “externally” with respect to the plasmid.

It has been a further object of the invention to provide a host vector system that combines control of PCN with antibiotic-free selection.

The present invention relates to a host-vector system comprising a non-naturally occurring bacterial host cell and a plasmid, wherein said plasmid has a ColE1-type origin of replication, wherein said bacterial host cell contains, integrated in its genome under the control of an inducible promoter, a DNA sequence encoding an RNA molecule that is able to interact with and inhibit a plasmid-transcribed RNA molecule, thereby controlling plasmid replication, wherein said RNA molecule is selected from
a) an RNA molecule that interacts with plasmid-transcribed RNAI, whereby, upon induction of said promoter and transcription of said DNA sequence, replication of the plasmid is upregulated;
b) an RNA molecule that interacts with plasmid-transcribed RNAII, whereby, upon induction of the promoter and transcription of said DNA sequence, replication of said plasmid is downregulated; and wherein

in the case of using an RNA molecule defined in b), said plasmid's ColE1 origin of replication is mutated such that the function of the RNAI promoter is abolished or significantly reduced.

When using the host-vector system of the invention in a fermentation process, plasmid copy number (PCN) can be controlled by regulating transcription of the genome-encoded RNA molecule that increases (a) or decreases (b) PCN, whereby the metabolic load during accumulation of biomass can be minimized. This is achieved by inducing the promoter at a late stage of the fermentation process in embodiment a), while inducing early on during fermentation and silencing the promoter towards the end of fermentation according to embodiment b).

The term “non naturally” in context with a bacterial host strain according to the invention means any genetically modified bacterial host strain not occurring in nature while having the ability to replicate in ColE1 plasmids an DNA sequence integrated to its genome (e.g. by means of recombinant techniques) which encodes an RNA molecule that is able to interact with and inhibit a plasmid-transcribed RNA molecule that controls plasmid replication.

The term “plasmid-transcribed” or “plasmid-derived” in the context with RNAI or RNAII, if not otherwise stated, designates RNAI or RNAII transcribed from the plasmid's ColE1 origin of replication.

The term “able to interact” defines the property of an RNA molecule to bind to said plasmid-transcribed RNA molecule such that its function is blocked.

The term “ColE1-type origin of replication” refers to a wild-type ColE1 origin of replication or a mutated version thereof, as defined herein.

The term “significantly” in context of “significantly reduced” function of the RNAI promoter means a reduction rate of RNAI expression in the plasmid according to the invention comprising genetically modified RNAI promoter by ca. 30%, preferably by ca. 50% most preferably by ca. 70% when compared to RNAI expression in the non modified (original) plasmid origin of the replication.

The term “RNA structure” means, if not otherwise stated, any 3-dimensional RNA II structure that maintains both the ability of its interaction with RNA I resulting in downregulation of the plasmid according to the invention and its functionality as a primer resulting in the plasmid replication.

The RNA molecule that is able to interact with said plasmid-transcribed RNA molecule and thereby has the ability to regulate replication of the ColE1 plasmid and, consequently, the PCN, is referred to as “PCN control sequence” or “PCN control molecule”. (For simplicity, this term is used both for the RNA sequence and for the DNA sequence encoding it, the latter both when inserted or for insertion into the host cell's genome).

In the embodiment of the invention as defined in a), said PCN control DNA sequence encodes an RNA molecule that interacts with and thereby inhibits the function of plasmid-transcribed RNAI. Such embodiment is based on the fact that interaction with RNAI leads to decreased amounts of free RNAI, which results in decreased amounts of replication inhibitor and, consequently, to increased replication of plasmid. In this embodiment, induction is done late in the fermentation process. In this context, “late induction” means that induction occurs approximately at or after half of the overall fermentation period, i.e. ca. at the end of after half of the number of generations. For example, if fermentation lasts ca. 28 hrs and involves four generations, induction is done ca. at the end of or after two generations.

According to this embodiment, the compound used for inducing transcription (i.e. the inducer) may, but need not be degradable/metabolizable, e.g. IPTG.

In certain aspects of this embodiment, the PCN control DNA sequence that inhibits plasmid-derived RNAI is a sequence that encodes wild-type RNAII, or, in the case that the plasmid-encoded RNAI contains modification(s), e.g. is present as a reverse or complementary sequence and/or contains one or more mutation(s), it is an RNAII sequence that is modified in a corresponding manner. The RNAII sequence, wild-type or modified, may also be truncated such that at least two of the three naturally occurring loops, either loop 1 and 2, or loop 2 and 3, or loop 1 and 3 are present.

According to another aspect of embodiment a), the PCN control molecule that inhibits plasmid-transcribed RNAI is a tRNA molecule (Wang et al., 2006; Wrobel et al., 1998).

This embodiment makes use of the RNA-based copy number control mechanism of ColE1-type plasmids and the interaction of said copy number control mechanism with uncharged tRNAs. It has been shown that overexpression of the alanine tRNA (anticodon UGC) induces cleavage of RNAI and results in an increase in ColE1-like plasmid DNA copy number (Wang et al., 2006), the suggested mode of action being the interaction of the uncharged form of said tRNA with the RNAI molecule.

Thus, in order to be able to inhibit plasmid-transcribed RNAI, the PCN control DNA sequence encodes a tRNA that is modified, due to mutations, in the acceptor stem such that the tRNA is only inefficiently charged with amino acids (i.e. the amino acid is not or inefficiently attached to its cognate tRNA by an aminoacyl-tRNA synthetase) and thus remains primarily un-loaded (Beuning et al., 2002). By inducing the promoter that controls expression of such mutated tRNA, interaction with and thus inhibition of RNAI occurs and replication increases.

By way of example, the PCN control DNA encodes the AlaU tRNA (Alanyl-tRNA-1B; Genbank Accession No. K00140), which has a nucleotide transversion at the 2:71 base pair position (G2:C71 to C2:G71), as described by Beuning et al., 2002.

Likewise, other tRNAs that have the ability to interact with RNAI can be appropriately modified to serve as PCN control sequences; after modification of the wild-type acceptor stem according to the principle and methods as described by Beuning et al., 2002, the mutated sequences (point mutations, insertional or deletion mutants) can be tested by cloning them, under control of an inducible or constitutive promoter, into a test plasmid, which may be, but not necessarily, a ColE1 plasmid, and determine whether the mutations have an effect by increasing or decreasing plasmid copy number.

In a further aspect of embodiment a), the PCN control molecule is a ribozyme-type RNA that recognizes and binds to plasmid-derived RNAI.

Ribozymes are antisense RNA molecules that have catalytic activity. They function by binding to the target RNA moiety through Watson-Crick base pairing and inactivate it by cleaving the phosphodiester backbone at a specific cutting site. The flanking arms of the ribozyme that bind to the substrate RNA may range between 6 and 12 nucleotides, the cleavage site between the flanking arms is UH, where U is Uracil and H is

Uracil, Adenin or Cytosin (Amarzguioui and Prydz, 1998).

FIG. 1a shows the generic design of a hammerhead ribozyme, wherein a naturally occurring UH cleavage site (uridine (U) followed by a C, A, or U) is located within the RNAI sequence. To identify a suitable ribozyme, the skilled person can design ribozyme constructs directed against different cleavage sites of RNAI and screen them in an vitro ribozyme cleavage assay, e.g. as described by Jarvis et al., 1996. An example for such a ribozyme construct is shown in FIG. 1b. The potential UH sites in the RNAI encoding DNA sequence (see also SEQ ID NO: 1) are in bold and underlined.

According to another aspect of embodiment a), the PCN control sequence that effects plasmid replication by interacting with plasmid-transcribed RNAI is an anti-eutE (ethanolamine utilization protein) sequence. It is shown by Sarkar et al., 2002, that an anti-eutE sequence that is in the reverse orientation of the eutE gene (Genbank Accession No. AE014075; region 2841106 . . . 2842509) and starts at 717 nt from the eutE start codon, is able to interact with RNAI and thus has the potential to increase plasmid synthesis. This sequence, which has a homology of 15 out of 16 nt with RNAI, may be modified to be more or less homologous with RNAI (e.g. 16/16 instead of 15/16, or 14/16 instead of 15/16).

According to embodiment b), said PCN control sequence encodes RNA that interacts with and inhibits plasmid-derived RNAII. Since RNAII is the molecule that initiates plasmid replication, according to this embodiment, plasmid replication is inhibited. In this embodiment, induction is done at the beginning and terminated towards the end of the fermentation process, i.e. after half of the overall fermentation period (e.g. after two out of four generations or after ca. 5-7 generations in the case that the overall fermentation comprises 10-15 fermentations). Interaction of the PCN control molecule, which is transcribed from the host genome throughout most of the fermentation period, with the plasmid-transcribed RNAII diminishes replication and keeps the PCN low. The degree of inhibition can be controlled either by using promoters with different strength or by decreasing the homology of the PCN control sequence to its RNAII target. In this embodiment, the inducer is preferably degradable and its amount is calculated such that it has been degraded by half of the fermentation period.

Examples of inducers are lactose or arabinose; since they are biodegradable and allow for tightly regulating expression of the PCN control molecule, they are usually preferred. Specific control of the lac promoter or the ara promoter depends on the availability of the corresponding carbohydrate in the growth media. Lactose binds to lacI, which is the repressor for the lac operator. If lactose is missing from the growth medium, the repressor binds very tightly to the lac operator sequence, and thereby prevents transcription from said promoter. When cells are grown in the presence of lactose, a lactose metabolite, allolactose, binds to the repressor, causing conformational changes that prevent the repressor from binding to the operator. Thus the altered repressor is unable to prevent transcription from the lac promoter (Reznikoff, 1992).

In the case of arabinose, positive regulation is used instead of negative regulation. If arabinose is present, arabinose binds to the AraC protein. This complex allows RNA polymerase to bind to the promoter. If arabinose is absent, the AraC assumes a different conformation that binds to the ara1 and ara0 region and thereby prevents the transcription of said promoter (Schleif et al., 2000).

Alternatively to using a degradable inducer, an inducer may be used that can be inactivated by some other mechanism, e.g. by addition of substances that specifically inhibit induction, e.g. glucose: Both the lactose promoter (pLac) and the arabinose promoter (pBad) provide only a very low expression level when glucose is present in the growth medium. For high expression from these promoters, it is essential that glucose is absent from the medium, inducing the formation of cAMP. cAMP binds to cAMP receptor protein (CRP) and this complex further binds to operator sequences in the pLac or the pBad.

According to embodiment b), PCN control sequences that interact with plasmid-transcribed RNAII may be selected from: (i) RNAI, (ii) parts of RNAI, (iii) mutants of RNAI that are directed to correspondingly mutated RNAII, preferably with mutations within one or more loops that do not change the structure of the RNA (e.g. by being complementary but not reverse; Grabherr et al., 2002; WO 02/29067).

In embodiment b), it needs to be ensured that PCN is exclusively regulated by the PCN control sequence that is transcribed, under the control of an inducible promoter, from the host's genome, whereby the inducer is metabolizable/degradable or can be inactivated, as herein described. Exclusive control of PCN by the PCN control sequence, i.e. without influence of the plasmid, is achieved by silencing RNAI transcription from the plasmid such that there is no translation of RNAI from the plasmid.

According to embodiment b) the host vector system of the invention therefore contains a plasmid in which the ColE1 origin of replication is mutated such that the function of the RNAI promoter is abolished (or significantly reduced, e.g. by deleting the −35 box only), while the function of RNAII remains essentially unchanged. Since RNAI and RNAII are encoded in antisense, it has to be ensured that deletion of RNAI promoter activity does not, or only to a minor extent, effect the structure of RNAII. By way of example, this can be achieved by point mutations in the −35 and/or −10 consensus sequence of the RNAI promoter. Any mutation may be made that does not change the RNAII structure but abolishes the activity of the RNAI promoter, which can be achieved by using the complementary, but not reverse sequence. Such plasmid is also subject of the present invention.

FIG. 2 shows mutations of the RNAII promoter that adjust the sequence to commonly used, highly active promoters in E. coli (Makrides, 1996; (SEQ ID NO: 2: wildtype sequence; SEQ ID NO: 3: mutated sequence).

According to embodiment b), a metabolizable (degradable) inducer is present from the beginning of the fermentation process such that the promoter is active during most of the fermentation period, whereby the amount of inducer, which is either a component of the medium or added at the beginning of fermentation, is such that it decreases over fermentation and is used up late in the fermentation process, i.e. ca. at of after half of the fermentation period. This has the consequence that, while the inducer is present, the PCN control molecule is transcribed from the genome and interacts with plasmid-transcribed RNAII. This results in a low PCN and a low metabolic load. When, towards the end of the fermentation process, the inducer is used up, then transcription of the PCN control sequence stops, which results in an increase of PCN.

Higher levels of RNAII can further be achieved by replacing the RNAII promoter by a stronger and/or an inducible promoter, e.g. the RNAI promoter, which leads to a 5-fold increase in transcription (Lin-Chao et al., 1987). A mutation resulting in an increase of RNAII transcription may be present on the plasmid by itself, or, optionally, in addition to the mutation that abolishes the function of the RNAI promoter in the case that abolishment of RNAI promoter function is not complete or in the case that, although this is not desirable, such mutation of the RNAI promoter does, to a certain extent, impair the folding and function of RNAII.

In a further aspect, the invention relates to a non-naturally occurring bacterial host cell in which a plasmid with a ColE1-type origin of replication can be replicated, wherein said bacterial host cell contains, integrated in its genome, a DNA sequence encoding an RNA molecule that has the ability to interact with RNAI or RNAII transcribed from a plasmid with a ColE1-type origin, when such plasmid is present in the host cell, wherein transcription of said DNA sequence is under the control of an inducible promoter, with the proviso that said DNA sequence exclusively regulates plasmid replication without being operably linked to a functional DNA sequence on the genome. According to this aspect, the RNA molecules with the ability to interact with plasmid-derived RNAI or RNAII with a ColE1-type origin of replication have the meanings given above for embodiments a) or b).

In another embodiment, the host-vector system of the invention is extended to combine PCN control with antibiotic-free selection. This embodiment combines the system for antibiotic free selection based on RNA-RNA interaction with an inducible plasmid host-vector system. This embodiment makes use of an artificial RNA-based antisense mechanism that mimics the naturally occurring ColE1-type copy number control mechanism, in order to regulate the expression of one or more toxic or lethal genes that are present in the bacterial host cell, preferably inserted in the bacterial genome and serve as selection marker (Mairhofer et al., 2008; WO 2006/029985).

According to this aspect, the present invention relates to a host-vector system comprising a non-naturally occurring bacterial host cell and a plasmid with a ColE1-type origin of replication, wherein said bacterial host cell contains, integrated in its genome
i) a DNA sequence, under the control of an inducible promoter, encoding a first RNA molecule that is able to interact with and inhibit a plasmid-transcribed RNA molecule, thereby controlling plasmid replication, wherein said first RNA molecule is selected from
   a) an RNA molecule that interacts with plasmid-transcribed RNAI;
   b) an RNA molecule that interacts with plasmid-transcribed RNAII;
ii) a DNA sequence encoding a protein that is lethal or toxic to said cell, and, operably associated thereto,
iii) a DNA sequence encoding a second RNA molecule that has the ability to interact with a third RNA molecule that mimics RNAI and that is transcribed from said plasmid, and wherein

said plasmid contains
i) at a locus other than the ColE1-type origin of replication, a sequence, under the control of a promoter, that encodes said third RNA molecule that mimics RNAI, and
ii) in the case of using an RNA molecule defined in b), a mutated ColE1 origin of replication such that the function of the RNAI promoter is abolished or significantly reduced;
   whereby, in the presence of said plasmid, said third RNAI-mimicking molecule interacts with second RNA molecule such that expression of said toxic protein is prevented and whereby, in the absence of said plasmid, said toxic protein is expressed, and whereby, upon induction of the promoter and transcription of said first RNA molecule, replication of said plasmid is upregulated in the case of a) or downregulated in the case of b).

The meanings of said first RNA molecules interacting with plasmid-transcribed RNAI or RNAII are those given above for the PCN control sequences.

The third RNA molecule (“RNA molecule that mimics RNAI”) is not the “wild-type” RNAI molecule derived from the origin of replication of the ColE1 plasmid, or a part thereof, but an “RNAI-like molecule”. This molecule mimics the structure of at least two loops of RNAI, either loop 1 and 2, loop 2 and 3, loop 1 and 3 or loop 1, 2 and 3. Said RNA preferably consists of the complementary, but not reverse sequence of RNAI or parts thereof. By changing each nucleotide into its complement, e.g. A to T, T to A, C to G, G to C, the sequence is different in that it is complementary but not reverse, while the RNA structure remains unchanged.

The RNAI-mimicking molecule encoded by the plasmid functions as an antisense molecule in that it interacts with said second RNA molecule that is operably linked to the RNA which encodes the lethal or toxic protein and thus abolishes translation thereof. Said protein (in the following “the toxic protein”; the DNA encoding it “the toxic gene”) is either toxic or lethal per se to the cell or it represses an essential gene product and thereby causes cell death. Interaction of the plasmid-derived RNAI-mimicking molecule with said second RNA molecule that is operably linked to said toxic gene is therefore required for the cell to survive. Said toxic gene is under control of a promoter, preferably one that can be tightly regulated.

The second RNA molecule that has the ability to interact with said third RNAI-mimicking molecule is a molecule that mimics an RNAII molecule, i.e. an RNAII-like molecule (as defined in WO 2006/029985)that is complementary to said RNAI-like molecule transcribed from the plasmid.

As distinguished from the antibiotic-free selection system described in WO 2006/029985, it is not the RNAI molecule derived from the ColE1 origin of replication that functions as the antisense molecule for the RNAII-like molecule that is operably linked to the RNA transcribed from the genome that encodes the toxic gene, but an RNAI-like artificial molecule that is transcribed from the backbone of the plasmid (i.e. from a locus other than the ori), under the control of a promoter, preferably a constitutive promoter which preferably has similar transcriptional activity as the RNAI promoter or is identical with the RNAI promoter. Since, according to embodiment b), the RNAI promoter has been inactivated, there is no wild-type RNAI transcribed from the plasmid. The RNAI-like molecule, in this embodiment transcribed from the plasmid backbone, binds to its complementary sequence, which is operably linked to the toxic transcript as described WO 2006/029985. Preferably, the RNAI-mimicking artificial molecule is partially complementary, but not reverse to the naturally occurring RNAI sequence. “Partially complementary” preferably means that loop III is the native loop III of RNAI, which is maintained because it acts as a terminator signal for transcription. Alternatively, if termination is not inherent to the sequence (as it is in the case that loop III, the native RNAI-terminator is present), other termination elements may be used, e.g. the T7 terminator in case the T7-promoter is used for transcription.

RNAI that functions as the PCN control sequence is exclusively transcribed from the genome under control of an inducible promoter.

Genes suitable as toxic genes for the present invention are described in WO 2006/029985. The toxic gene encodes a protein that is lethal or toxic per se; however, in this embodiment, in the meaning of the present invention, the term “toxic gene” also encompasses genes the expression of which results in a toxic effect that is not directly due to the expression product, but is based on other mechanisms, e.g. generation of a toxic substance upon expression of the toxic gene.

In a preferred embodiment, the toxic protein is not lethal or toxic per se or due to a toxic effect generated upon its expression, but by repressing the transcription of a gene that is essential for growth of said bacterial cell. Such protein, or the DNA encoding it, respectively, is referred to as “repressor” or “repressor gene”, respectively, and the gene that is essential for growth of the bacterial cells is referred to as “essential gene”.

Transcription of the RNA encoding the lethal or toxic protein and the RNA operably linked thereto) is controlled by an inducible promoter, e.g. lac or the lacUV5 promoter, the p_{BAD} promoter (Guzman et al., 1995), the trp promoter (inhibited by tryptophan), the P₁ promoter (with cᵢ repressor) or the gal promoter are used. The toxic gene is preferably a repressor gene, e.g. the Tet-repressor gene which is targeted towards an essential gene. To this end, the essential gene is modified with respect to its transcriptional control, i.e. by insertion into the promoter of a corresponding operator which can be repressed by the repressor gene, e.g. the Tet-repressor. An example for an essential gene is the murA encoding gene (Mairhofer et al., 2008).

The above embodiment is a combination of the PCN control system with antibiotic-free selection. According to these aspects, useful PCN control sequences are those defined above. With regard to fermentation, e.g. use of inducers and time point of induction, the above-defined criteria for embodiments a) and b) apply.

In a further aspect, the present invention relates to methods for producing plasmid DNA, wherein an above-described host-vector system (with or without toxic gene integrated in the host genome for antibiotic-free selection) is cultivated and wherein, when an RNA molecule as defined in a) is used, the promoter is induced after half of the cultivation period, and wherein, when an RNA molecule as defined in b) is used, the promoter is induced early in the cultivation process and silenced at or after half of the cultivation period. For therapeutic applications, the plasmid contains the DNA sequence (the “gene of interest”) encoding the therapeutic protein of interest operably associated with a eukaryotic promoter for expression in the patient, e.g. the CMV promoter.

The term “after half of the cultivation period” means a time point wherein half of the number of generations of the host cells that are produced within the full cultivation period, is formed. A full cultivation period means a period calculated from the inoculation of the fermentation medium with a bacterial host cell according to the invention until the fermentation process is completed.

The term “protein of interest” means, if not otherwise stated, any recombinant protein that is expressed by ColE1 type of plasmid according to the invention. The protein can be expressed under the control of a promoter (inducible or constitutive) in a microbial cell and then to be used for different purposes, for example but not limited to use as an active ingredient of a biopharmaceutical, technical enzyme, diagnostic enzyme. The protein can also be expressed under the control of a promoter (inducible or constitutive) in a host organism (man, animal, etc) acting either as an active pharmaceutical ingredient in the host's cells or as an antigen of a vaccine if the plasmid is used for gene therapy or as a gene vaccine.

In addition to the production of plasmid DNA, the method of the invention is also useful for producing recombinant proteins. In such process, cells carrying a plasmid that also contains a sequence encoding the protein of interest under the control of a prokaryotic (inducible or constitutive) promoter are cultivated such that the protein is expressed; the parameters for fermentation (e.g. choice of inducers for PCN control sequence and time point of induction) are as described for plasmid production.

1. Host cells

Since their replication depends on the host machinery, ColE1-type plasmids are plasmids with a narrow host range. Replication is limited to E. coli and related bacteria such as Salmonella and Klebsiella (Kues, 1989). Thus, the only mandatory property of the host is that it has the ability to replicate ColE1 plasmids. Examples for suitable hosts are the widely used Escherichia coli strains K12 or the B strain or related commercially available strains, e.g JM108, TG1, DH5alpha, Nova Blue, X11 Blue, HMS174 or B121 (for review see Casali, 2003).

Preferred genetic features of the host cell are mutations that improve plasmid stability and quality or recovery of intact recombinant protein. Examples of desirable genetic traits are recA (absence of homologous recombination), endA (absence of endonuclease I activity, which improves the quality of plasmid minipreps) or ompT (absence of an outer membrane protease), hsdr (abolished restriction but not methylation of certain sequences), hsdS (abolished restriction and methylation of certain sequences).

In the experiments of the invention, the host strain HMS174(DE3) (Novagen) is used, which contains the DE3 phage with the IPTG inducible T7 polymerase in its genome (Studier and Moffatt, 1986) or JM109 (New England Biolabs), which contains the lacI^{q} gene for tight control of the lac promoter or derivatives thereof. Another example for a suitable host is HMS174(DE)pLysS, which additionally contains the pACYC184 plasmid (Cm^{R}) that carries the gene for the T7-lysozyme to decrease the transcriptional activity of the T7-Promoter in the un-induced state. Further hosts are K12 strains and BL21 strains and derivatives thereof, e.g. DH5alpha, JM108, BL21, BL21DE3.

Inducible promoters that may be used include the T7 promoter, araC promoter, lac promoter, tac promoter, trp promoter, all other promoters that contain the lac-operator, tet-operator or any other operator that can preferably be induced or repressed by degradable inducers such as arabinose, lactose, glucose, maltose, tryptophan etc.

2. Constructs for Engineering the Host Cells

The constructs to be introduced into the host genome may be obtained according to the methods described in WO 2006/029985. All the components—two homologous arms for recombination, promoter+operator [P+O], PCN control sequence, (optionally toxic gene plus sequence controlling toxic gene expression), with a transcriptional terminator and a resistance gene cassette, e.g. the Kan cassette (Kanamycin resistance cassette containing FRT, the +/−FLP recombinase recognition target sequences: alternatively, other conventional selection markers may be used in a corresponding manner) are cloned into the multiple cloning site of a suitable vector, e.g. pBluescript KS+. Linear fragments for genomic insertion are cut out with restriction enzymes or amplified by PCR.

The kanamycin resistance cassette can be obtained, by way of example, from the pUC4K vector (Invitrogen). It can be cloned into the construct at two different sites, namely in front of or behind the PCN control sequence, e.g. the RNAI sequence. To avoid unintended premature transcription of the PCN control sequence before it is turned on deliberately, the sequence is preferably inserted in the opposite direction as the chromosomal genes.

Preferably, the construct is introduced into the bacterial chromosome by conventional methods, by using linear fragments that contain flanking sequences homologous to a neutral site on the chromosome, for example to the attTN7-site (Rogers, 1986; Waddel and Craig, 1988; Craig, 1989) or to the recA site.

Fragments are transformed into the host, e.g. E. coli strains MG1655 or HMS174 that contain the plasmid pKD46 (Datsenko; 2000). This plasmid carries the λ Red function (γ, β, exo) that promotes recombination in vivo. Alternatively, DY378 (Yu, 2000), an E. coli K12 strain which carries the defective λ prophage, can be used. In case of MG1655 or DY378 the chromosomal locus including the expression fragment can be brought into the HMS174(DE3) genome via transduction by P1 phage. Positive clones are selected for antibiotic resistance, e.g. in the case of using the Kan cassette for kanamycin, or chloramphenicol. The resistance genes can be eliminated afterwards using the FLP recombinase function based on the site-specific recombination system of the yeast 2 micron plasmid, the FLP recombinase and its recombination target sites FRTs (Datsenko and Wanner, 2000).

Alternatively to having the construct integrated in the host's genome, it may be present on a phage or a plasmid that is different from a ColE1-type plasmid and that is compatible with the system of the invention in the sense that it does not influence expression of the PCN control sequence. Examples for suitable plasmids or phages are pACYC184 (which is a derivative of miniplasmid p15A; see Chang and Cohen, 1978),

R1-miniplasmids (Diaz and Staudenbauer, 1982), F1-based plasmids or filamentous phages (Lin, 1984) or the plasmid pMMB67EH (Fürste, 1986) that is used in the experiments of the invention.

3. Construction of Plasmids

As described above, for certain embodiments, ColE1 plasmids are used that are genetically modified in order to i) have the RNAI promoter mutated, thereby abolishing RNAI promoter function. In this context, it is essential that the structure and functionality of RNAII, which is partially complementary to the RNAI promoter, is maintained; ii) or have the RNAII promoter mutated in a way that promoter function is enhanced; iii) have the RNAII promoter replaced by the RNAI promoter, which is 5-fold stronger than the RNAII promoter. Suitable modified RNAII promoters can be identified using a promoter library containing randomized −10, 35 or −10 and −35 regions. The library can be screened for enhanced RNAII promoter function using antibiotic selection pressure, dependent of the antibiotic resistance gene encoded on the test-plasmid. Plasmids containing benefical RNAII promoter variants should be more resistant to high antibiotic concentrations.

Additionally, in certain embodiments, sequences can be inserted in the plasmid backbone, at a locus different from the origin of replication, e.g. upstream of the origin of replication, that produce RNAI that mimics the structure of RNAI or parts thereof, and thus serve to interact with an antisense-RNA, functionally coupled to the toxic gene derived from the host genome.

4. Fermentation

The present invention can be widely used in state-of-the-art fermentations, both for plasmid DNA production and for producing recombinant proteins.

Several methods for fermentation of pDNA have been described that are useful to be applied to the present invention. The methods for plasmid DNA production differ with regard to the level of control imposed upon the cells and the numerous factors that influence fermentation:

For pDNA production on a laboratory scale, cultivation of plasmid-bearing cells in shake flasks is the simplest method (e.g. Reinikainen et al., 1988).

To obtain higher quantities of plasmids, the cells can be cultivated in controlled fermenters in so-called “batch fermentations”, in which all nutrients are provided at the beginning and in which no nutrients are added during cultivation. Cultivations of this type may be carried out with culture media containing so called “complex components” as carbon and nitrogen sources, as described e.g. by O'Kennedy et al., 2003; and Lahijani et al., 1996; and in WO 96/40905; U.S. Pat. No. 5,487,986; WO 02/064752. Alternatively, synthetic media may be used for pDNA production, e.g. defined culture media that were specifically designed for pDNA production (Wang et al., 2001; WO 02/064752).

The present invention may also be used in fed batch fermentations of E. coli, in which one or more nutrients are supplied to the culture by feeding, typically by using a feed-back control algorithm by feeding nutrients in order to control a process parameter at a defined set point. Feed-back control is hence directly related to cell activities throughout fermentation. Control parameters which may be used for feed-back control of fermentations include pH value, on line measured cell density or dissolved oxygen tension (DOT). A feed-back algorithm for controlling the dissolved oxygen tension at a defined set point by the feeding rate is described in WO 99/61633.

Another, more complex algorithm uses both the DOT and the pH value as control parameters for a feed-back cultivation method (U.S. Pat. No. 5,955,323; Chen et al., 1997)

Another feeding mode is based on the supply of feeding medium following an exponential function. The feeding rate is controlled based on a desired specific growth rate μ. WO 96/40905 and O'Kennedy et al., 2003, describe methods that use an exponential fed-batch process for plasmid DNA production. Lahijani et al., 1996, describe combining exponential feeding with temperature-controllable enhancement of plasmid replication.

Alternatively, the invention may be applied in a process for producing plasmid DNA, in which E. coli cells are first grown in a pre culture and subsequently fermented in a main culture, the main culture being a fed-batch process comprising a batch phase and a feeding phase. The culture media of the batch phase and the culture medium added during the feeding phase are chemically defined, and the culture medium of the feeding phase contains a growth-limiting substrate and is added at a feeding rate that follows a pre-defined exponential function, thereby controlling the specific growth rate at a pre-defined value.

### BRIEF DESCRIPTION OF THE FIGURES

FIG. 1a: Generic design of a hammerhead ribozyme for RNAI cleavage

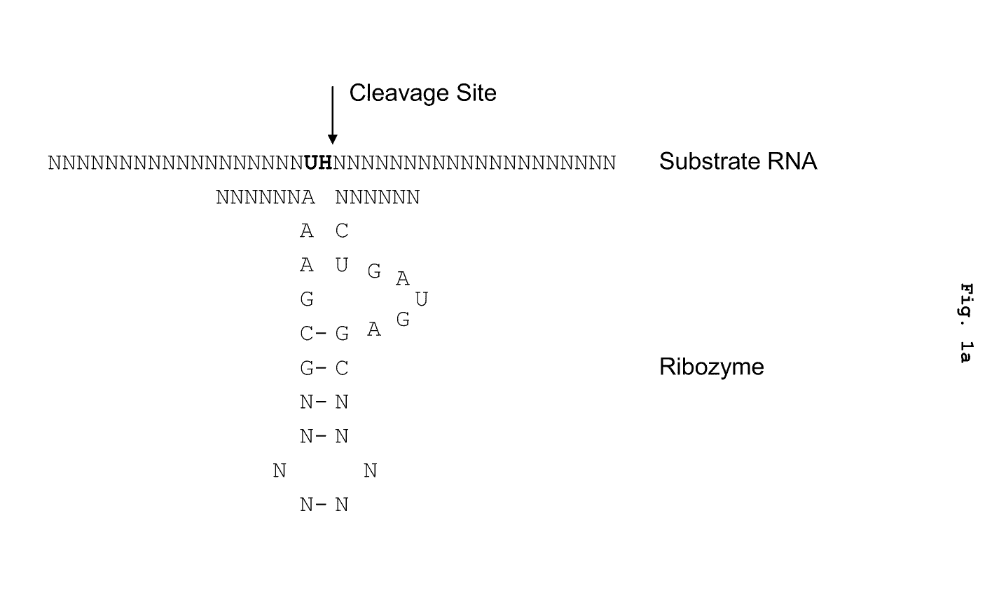

FIG. 1b: Ribozyme construct for RNAI cleavage

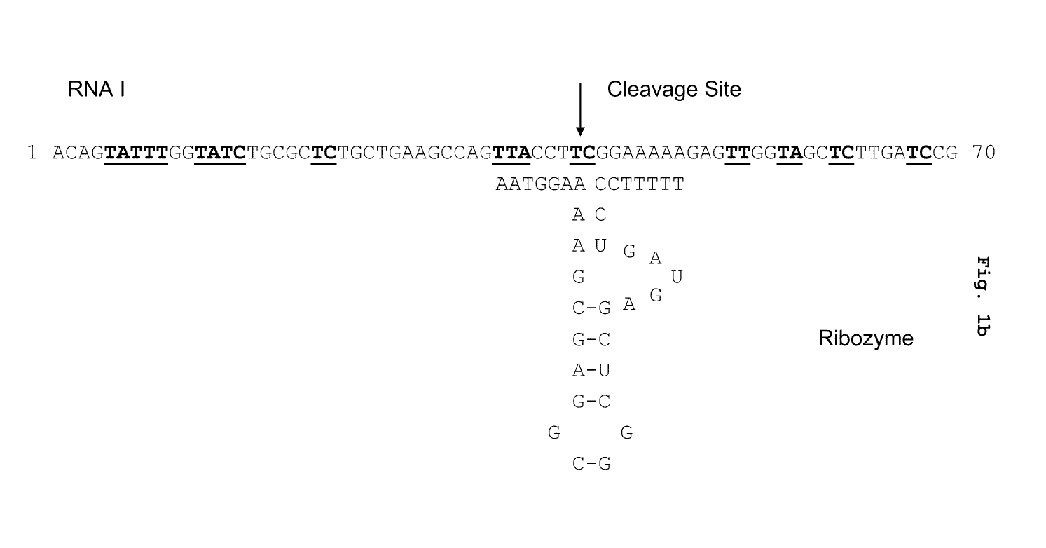

FIG. 2: Mutations of RNAII promoter that adjust the sequence to highly active promoters in E. coli

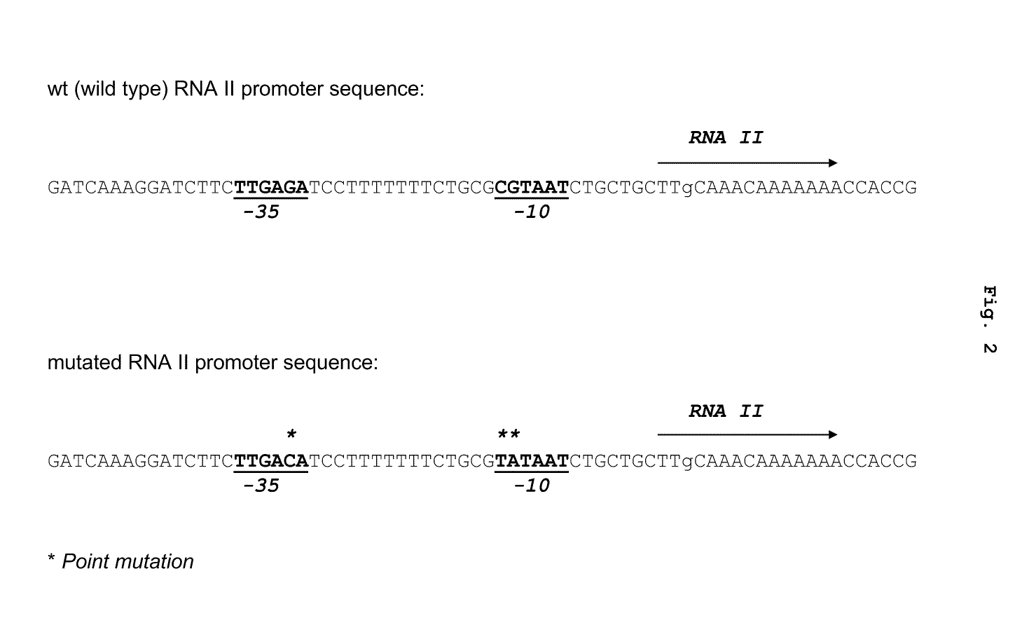

FIG. 3a: Schematic illustration of PCN control by inhibiting plasmid-derived RNAII by RNAI transcribed from the host's genome.

FIG. 3b: Expression cassette for chromosomal integration of an RNAI sequence for control of plasmid copy number

FIG. 4: Annotation of wild-type and mutated RNAI promoter sequence

FIG. 5: Gel electrophoresis of RNAI-promoter deleted pUC19 plasmids from cultivation of JM109 and JM1091acRNAI under induced (IPTG) and non-induced conditions

FIG. 6a: Schematic illustration of PCN control by inhibiting plasmid-derived RNAI by tRNA transcribed from the host's genome

FIG. 6b: Annotation of wild-type AlaU tRNA and mutated AlaU tRNA

FIG. 7: Schematic illustration of PCN control combined with antibiotic-free selection

FIG. 8: Plasmid containing RNAI-like sequence for plasmid-controlled silencing of a toxic gene

### EXAMPLE 1

PCN Control by Inhibiting Plasmid-Derived RNAII by RNAI Transcribed from the Host's Genome.

The experiments of this Example, which exemplify embodiment b) of the PCN control system, show uncoupling of RNAI and RNAII transcription by preventing transcription of plasmid-encoded RNAI and providing RNAI from the host genome. Abolishment of

RNAI transcription is achieved by the introduction of mutations in the RNAI promoter. Said mutations refer to point mutations in the −10 and −35 box of the RNAI promoter, that do not, or only insignificantly, alter the structure of RNAII (which has to be considered since RNAI and RNAII are encoded in anti-sense), but do abolish the function of the RNAI promoter.

The chromosome-encoded inducible RNAI or parts thereof allows normal growth of the host cell, due to lowering the metabolic load caused by over-replication. Due to the fact that the inducer is metabolized, transcription of the gene encoding RNAI or parts thereof is shut down at the very end of the bioprocess and plasmid DNA accumulates due to the lack of copy number control that is normally exerted by RNAI.

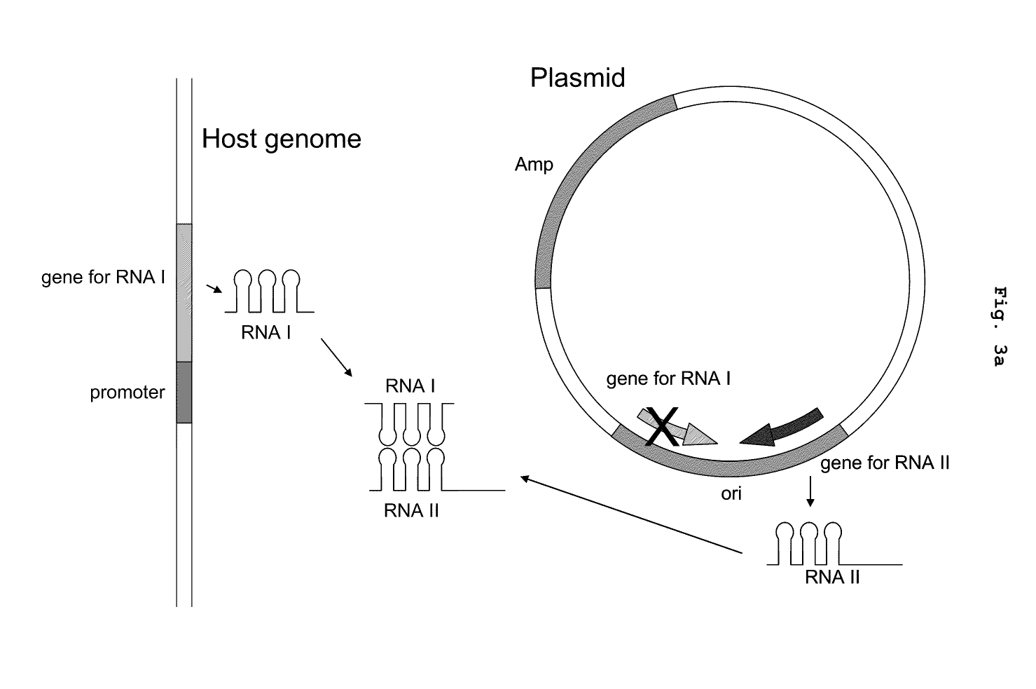

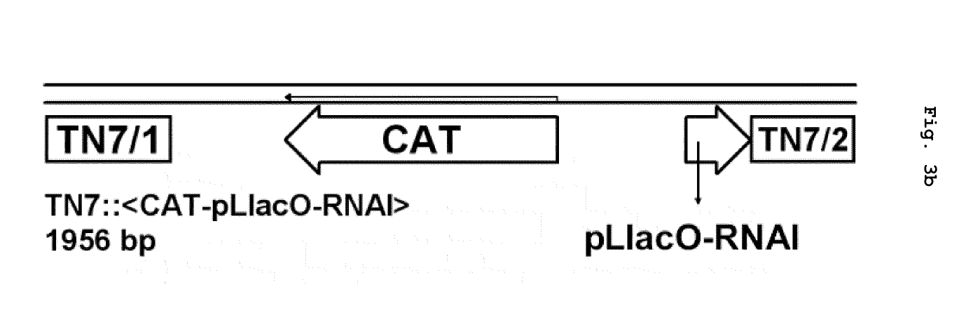

FIG. 3a schematically shows that the promoter function of RNAI in the origin of replication of the plasmid is abolished by targeted mutations, thus no RNAI is transcribed from the plasmid. A DNA sequence encoding RNAI under the control of an inducible promoter is located within the bacterial genome. When the inducer is present, RNAI is transcribed from the genome, binds to plasmid-derived RNAII and thereby down-regulates the PCN.

a) Construction of an RNAI Expression Host

Plasmid pBSK::TN7<CAT-PLlacO-1-RNAI>, acting as a source for creating linear DNA, is created using pre-made pBSK::TN7<CAT-T7-RNAI>. This pre-made plasmid is digested, using restriction enzymes BstI and NcoI, and the fragment containing the T7-RNAI sequence is gel-purified in order to obtain template DNA that only contains one copy of the RNAI gene. The PLlacO-1 is then amplified by PCR using the primers BlpI-pLlacO-back and XhoI-RNAI-for (see Table 1) and said linear DNA sequence as template. The pre-made pBSK::TN7<CAT-T7-RNAI>and pre-amplified PLlacO-1-RNAI are treated both with BlpI and XhoI, followed by ligation.

A linear DNA fragment is amplified from pBSK::TN7<CAT-PLlacO-1-RNAI>using primers TN7/1back and TN7/2for (see Table 1) and chromosomal integration of said DNA fragment (for schematic drawing see FIG. 3b), for sequence information see SEQ ID NO:8) into MG1655 is performed using the method described by Datsenko and Wanner (2000). The genetic modification is further transferred into recA− and lacIq+ host strain JM109 by Plvir transduction (transduction with lysogenic P1 phage; Sternberg and Hoess, 1983), yielding JM109::TN7<CAT-PLlacO-1-RNAI>.

TABLE 1
Primer List

| SEQ ID | | |
|---|---|---|
| NO: | Primer/Oligo | Sequence |
| 25 | B1pI-pLlacO- | ATGATGGCTAAGCATAAATGTGAGCGGATA |
| | back | ACATTGACATTGTGAGCGGATAACAAGATA |
| | | CTGAGCACACAGTATTTGGTATCTGCGC |
| 26 | XhoI-RNAI-for | CCGCTCGAGAACAAAAAAACCACCGCTACC |
| 27 | TN7/1back | GTTGCGACGGTGGTACG |
| 28 | TN7/2for | TGAAGAAGTTCGCGCGCG |

SEQ ID NO: 8 shows the sequence of cassette for RNAI expression that is integrated into the bacterial genome (1927 bp).

b) Generation of RNAI Promoter-Deleted pUC19

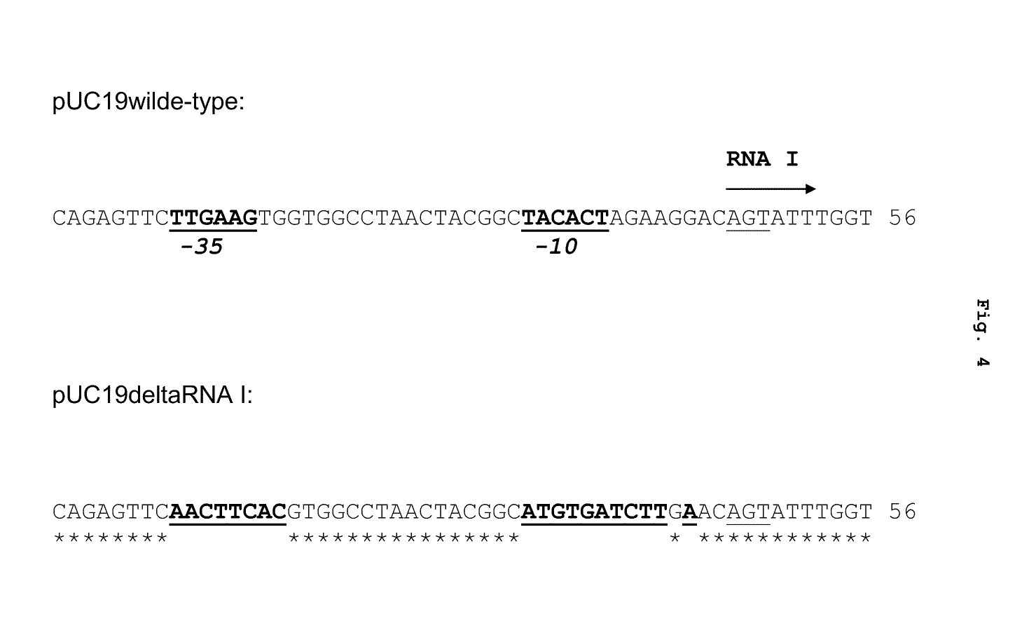

The RNAI-promoter-deleted variant of pUC19, referred to as pUC19ΔRNAI, is generated by inverse PCR using primers RNAI-l0promΔback and RNAI-35promΔfor (see Table 2), using pUC19 as template. The PCR product is further treated with polynucleotide kinase, followed by ligation. Annotation of the original pUC19 RNAI promoter site and sequence comparison of wt (SEQ ID NO: 4) and mutated (SEQ ID NO: 5) pUC19ARNAI sequence is shown in FIG. 4. Since RNAI and RNAII are encoded in antisense, mutagenesis of the −10 and −35 region of the RNAI promoter is performed in a way that does not impair the proper folding of RNAII.

TABLE 2
Primer List

| SEQ ID | | |
|---|---|---|
| NO: | Primer/Oligo | Sequence |
| 9 | RNAI- | CTACGGCATGTGATCTTGAACAGTATTTG |
| | 10promΔback | GTATCTGCGC |
| 10 | RNAI- | TTAGGCCACGTGAAGTTGAACTCTGTAGC |
| | 35promΔfor | ACCGCCTAC |

c) Cultivation of Host Cells Containing RNAI-Promoter Deleted pUC19

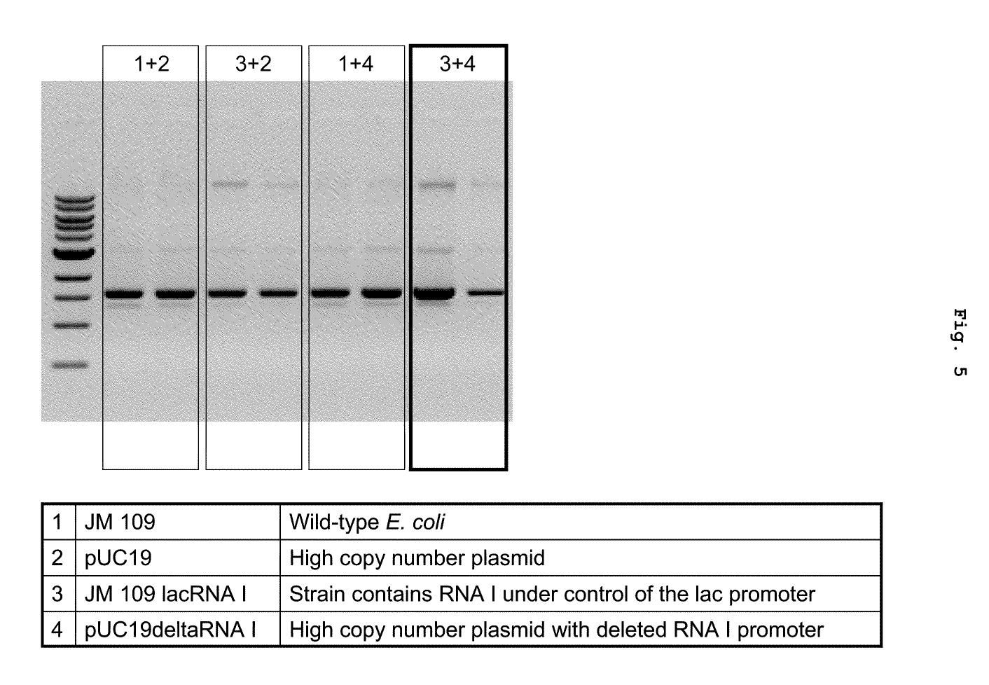

Plasmid replication of the plasmids pUC19 and pUC19deltaRNAI is tested with and without the addition of IPTG in JM109::TN7<CAT-PLlacO-1-RNAI>. After over-night cultivation in shake-flasks, plasmids are quantitatively isolated, set in relation to cell density and subjected to gel electrophoresis (FIG. 5). The final yield of DNA is determined spectrophotometrically (Table 3).

TABLE 3
yield of plasmids pUC19 and pUC19deltaRNAI from hosts JM109
and JM1091acRNAI under induced (IPTG) and non-induced culture
conditions, as determined spectrophotometrically.

| | Minipreps |
|---|---|
| JM109-lacRNAI | |
| +puc19 | 89.5 ng/μl |
| +puc19 + IPTG | 71.5 ng/μl |
| +puc19deltaRNAI | 198 ng/μl |
| +deltaRNAI + IPTG | 59.5 ng/μl |
| JM109 | |
| +puc19 | 102 ng/μl |
| +puc19 + IPTG | 115 ng/μl |
| +puc19deltaRNAI | 107 ng/μl |
| +deltaRNAI + IPTG | 119 ng/μl |

It is shown that the amount of RNAI and, hence, the PCN can be regulated externally by the addition of an appropriate inducer. Specifically, it is shown that plasmid pUC19deltaRNAI can be produced in JM109::TN7<CAT-pLₗₐₒ₋₁-RNAI>in sufficient amounts and that by addition of the inducer IPTG the plasmid copy number can be decreased (FIG. 5). Since no such effect can be seen for JM109, it can be attributed to the transcription of RNAI from the genome, which inhibits replication. Thus, this experiment exemplifies a plasmid production system that can be externally regulated.

In a fed-batch fermentation, IPTG is replaced by lactose, which is an inducer that is metabolized by the host. Cells are grown on a lactose batch medium, providing repression of plasmid replication due to production of RNAI. During the second phase of the bioprocess, cells are grown on a decreasing combined glucose/lactose feed, wherein 100 μmol/g BDM lactose is added to the feed medium, in addition to glucose (Striedner et al., 2003). Plasmid replication is induced in phase three of the bioprocess, whereby the lactose/glucose feed is shifted to a pure glucose feed which represses expression of RNAI.

### EXAMPLE 2

PCN Control by Inhibiting Plasmid-Derived RNAI by a Mutated tRNA Transcribed from the Host's Genome

Object of these experiments, which exemplify embodiment a) of the invention, is to provide, in the host's genome, an inducible tRNA molecule that, by point mutations introduced into the acceptor stem, is inefficiently charged with amino acids and thus remains essentially unloaded. By inducing the promoter that controls expression of said tRNA, inhibition of plasmid-derived RNAI occurs and replication increases. In this example, the inducer is being added later in the fed-batch process and does need to be metabolized by the host.

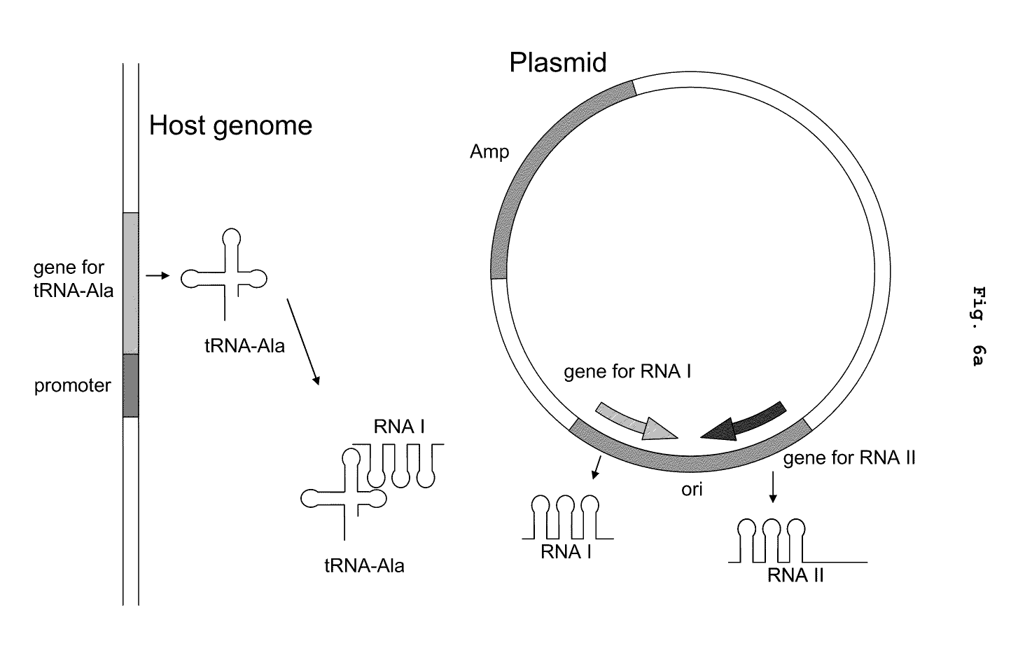

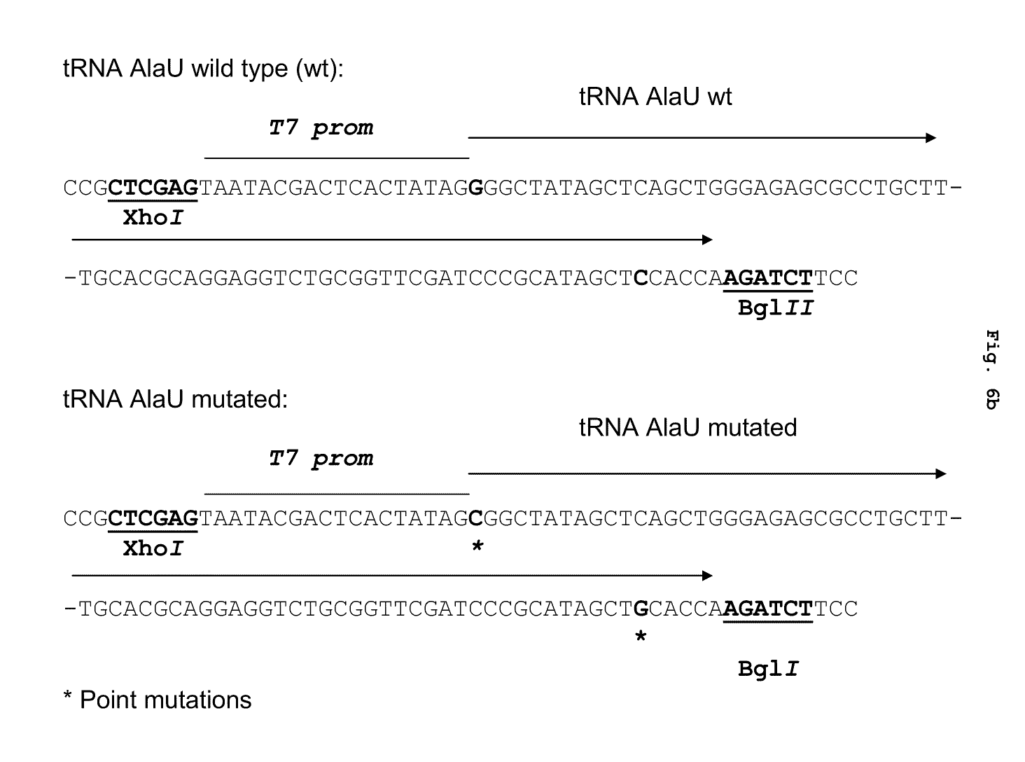

FIG. 6a schematically shows this embodiment: A DNA sequence encoding a mutated version of the tRNA that is normally charged with alanine, is inserted in the bacterial chromosome and transcribed under the control of an inducible promoter. When the inducer is present, tRNA is transcribed and binds to RNAI derived from the plasmid's origin of replication, thereby preventing the plasmid replication control.

Overexpression of an Escherichia coli tRNA from the Alanine Family

Plasmid pBSK::TN7<CAT-MCS> is created using pBSK::TN7<CAT-T7-GFP> as source plasmid and primers MCS-TN7-GFP2435bp-back and MCS-TN7-GFP1500bp-for (see Table 4) for PCR amplification. The resulting PCR product is digested with BamHI and ligated, yielding pBSK::TN7<CAT-MCS>.

Oligos XhoI-T7-AlaU-mut-back and BglII-tRNA-AlaU-mut-for or XhoI-T7-AlaU-org-back and BglII-tRNA-AlaU-org_for (see Table 1) are annealed in a thermal cycler following this profile: (i) heat to 95° C. and remain at 95° C. for 2 minutes, (ii) ramp cool to 25° C. over a period of 45 minutes, (iii) proceed to a storage temperature of 4° C. Annealed oligonucelotides are further treated with DNA polymerase I Large Fragment to create double stranded DNA. Subsequently, the insert containing the tRNA and vector pBSK::TN7<CAT-MCS> are treated with XhoI and BglII, followed by ligation, yielding plasmid pBSK::TN7<CAT-T7-tRNA1a-mut> or pBSK::TN7<CAT-T7-tRNA1a-org>. A linear DNA fragment is amplified from both plasmids using primers TN7/1back and TN7/2for (Table 4) and chromosomal integration of said DNA fragment (for schematic drawing see FIG. 6a, for sequence information see SEQ ID NO: 8) into MG1655 is performed using the method described by Datsenko and Wanner (2000). The genetic construct is further transferred into recA-host strain HMS174(DE3) by Plvir transduction, yielding HMS174(DE3)::TN7<CAT-T7-tRNA1a>.

For annotation of the wt tRNA AlaU sequence with the mutated sequence, see FIG. 6b; point mutations are indicated (SEQ ID NO: 6: tRNA AlaU wildtype, SEQ ID NO: 7: mutated version of tRNA AlaU).

TABLE 4
Primer List

| SEQ ID | | |
|---|---|---|
| NO: | Primer/Oligo | Sequence |
| 11 | MCS-TN7- | GCGCGGATCCCGGGCTCGAGGCCACTGGA |
| | GFP2435bp-back | GCACCTCAAAAAC |
| 12 | MCS-TN7- | GATGGGATCCAGATCTTCTAGAGCATCCA |
| | GFP1500bp-for | TTTATTACTCAACCG |
| 13 | XhoI-T7-AlaU- | CCGCTCGAGTAATACGACTCACTATAGCG |
| | mut-back | GCTATAGCTCAGCTGGGAGAGCGC |
| 14 | BglII-tRNA- | GGAAGATCTTGGTGCAGCTATGCGGGATC |
| | AlaU-mut-for | GAACCGCAGACCTCCTGCGTGCAAAGCAG |
| | | GCGCTCTCCCAGCTGAGCTA |
| 15 | XhoI-T7-AlaU- | CCGCTCGAGGTAGTTAATACGACTCACTA |
| | org-back | TAGGGGCTATAGCTCAGCTGGGAGAGCGC |
| 16 | BglII-tRNA- | GGAAGATCTTGGTGGAGCTATGCGGGATC |
| | AlaU-org-for | GAACCGCAGACCTCCTGCGTGCAAAGCAG |
| | | GCGCTCTCCCAGCTGAGCTA |

### EXAMPLE 3

Combination of PCN Control with Antibiotic-Free Selection

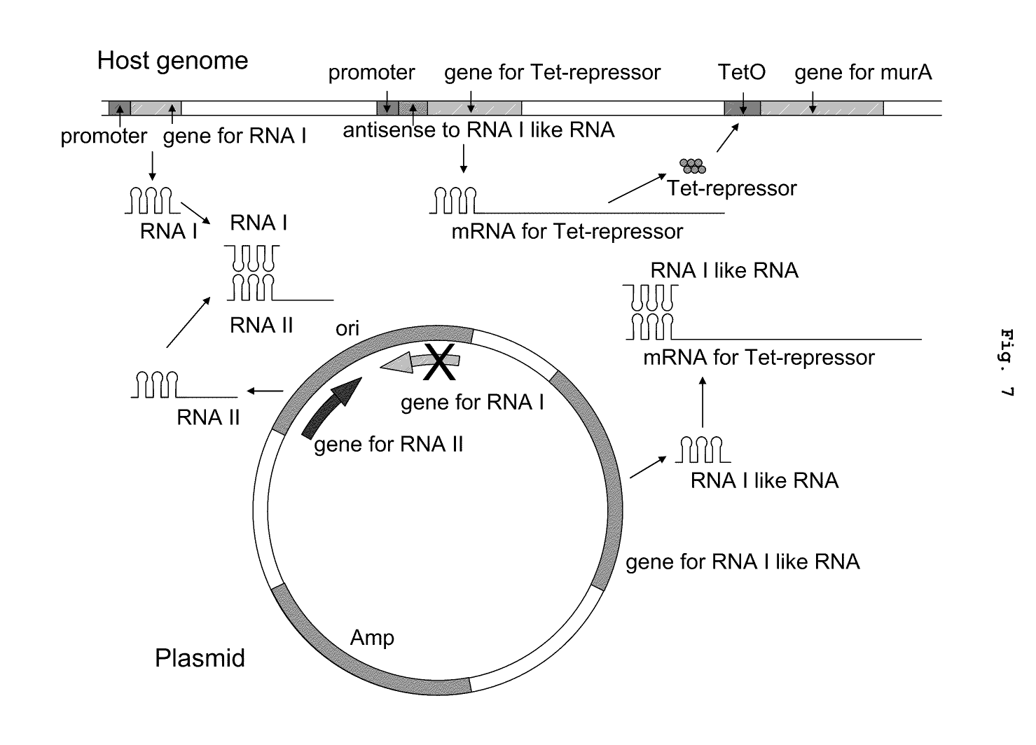

The PCN control system in this experiment corresponds to embodiment b). FIG. 7 schematically shows the set-up of this experiment: The promoter of RNAI on the plasmid is abolished by targeted point mutations to ensure that no RNAI is transcribed from the plasmid and PCN is exclusively controlled by the genome-encoded RNAI molecule. A sequence encoding RNAI is integrated in the bacterial chromosome and transcribed under control of an inducible promoter. When the inducer is present, RNAI is transcribed from the genome, binds to plasmid-derived RNAII, thereby controlling plasmid replication. In addition, an RNA molecule that mimics RNAI in that it has its structure, but is different in sequence (designated “RNAI-like” in FIG. 7), is transcribed, under control of an constitutive or inducible promoter, from a locus on the plasmid that is different from the ori. This RNAI-mimicking molecule binds to an mRNA derived from the chromosome that is operably linked to a sequence encoding a lethal or toxic protein, thus providing a selection mechanism for plasmid containing cells.

a) Construction of a Host/Vector System for Plasmid-Controlled Silencing of a Toxic Gene

In the tested construct, a DNA sequence encoding two RNA stem loops, containing the complementary sequence of Loop II and III of the naturally occurring RNAI (thus corresponding to the “RNAII-like molecule”, as defined in WO 2006/029985), is fused to the GFP sequence (which serves as a model sequence for the toxic gene) such that Int are incorporated between the RBS and the ATG-start codon, and such that the ATG is followed by AAT-codon (Asparagine) before the RNAII stem loop coding sequence starts. This sequence, under control of the T7 promoter, is inserted into the chromosome. A plasmid is constructed containing a tetracycline repressor (TetR) protein and a promoter pLtetO that drives the expression of an RNA, whose sequence is partially complementary, but not reverse to the naturally occurring RNAI sequence (partially complementary due to the fact that Loop III of this sequence is the native Loop III of RNA I, which is maintained because it acts as a terminator signal for transcription). The pLtetO is inducible by addition of anhydro-tetracycline (aTc) and the expression of GFP is silenced upon addition of this inducer, due to hybridization of the modified RNAI sequence that is partially complementary, but not reverse to the said stem loop structure.

b) Vector Construction pANTIGON

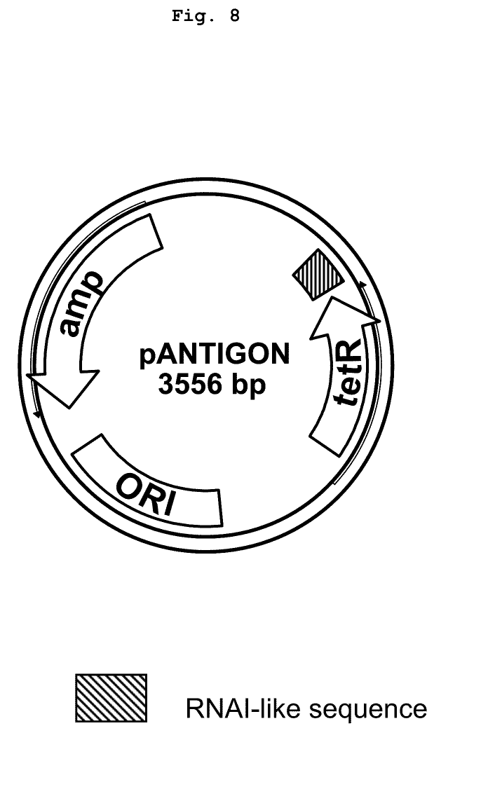

The gene encoding for the tetracycline repressor (TetR) is amplified from the tetracycline resistant strain HMS174(DE3)ilv-500::Tn10 containing Tn10, by primers NheI-tetR-back and BamHI-tetR-for (see Table 5) and cloned into XbaI/BamHI pre-digested pUC19. A functional promoter is provided by tetR-Prom-SalI-back and tetR-Prom-SalI-for by insertion into SalI restriction site of pUC19. The tet-inducible pLtetO-RNAst fusion is fully synthesized on the primers pLtetO-RNAst1-4. This RNA expression element is cloned into SmaI site of pUC19. Sequence of Loop III is later replaced for native Loop III of RNAI by PCR using primers RNAst-new-back and RNAst-new-for (see Table 1). PCR product is ligated and amplified. For a schematic drawing of pANTIGON, see FIG. 8.

c) Construction of an Expression Cassette for Chromosomal Integration

pBSK::TN7<CAT-T7-L23RNAst-GFP>is constructed using primers TN7-L3-GFP-back and TN7-RBS-L2-for (see Table 5), and pBSK::TN7<CAT-T7-L23-GFP>as template. TN7 expression cassette (for schematic drawing see FIG. 4) is amplified by TN7/1-back and TN7/2-for, plasmid template is digested by DpnI and linear DNA is used for genomic integration into MG1655 by the method described by Datsenko and Wanner. The construct is further transferred into recA-host strain HMS174(DE3) by Plvir transduction, yielding HMS174(DE3)::TN7<CAT-T7-L23RNAst-GFP>.

TABLE 5
Primer List

| Seq ID | | |
|---|---|---|
| NO: | Primer/Oligo | Sequence |
| 17 | NheI-tetR-back | GCTGCTGCTAGCATGATGTCTAGAT |
| | | TAGATAAAAG |
| 18 | BamHI-tetR-for | GCTGCTGGATCCTTAAGACCCACTT |
| | | TCACATTTAAG |
| 19 | tetR-Prom-SalI- | TCGATTTTCTCTATCACTGATAGGG |
| | back | AGTGGTAAAATAACTCTATCAATGA |
| | | TTAAGGAGG |
| 20 | tetR-Prom-SalI- | TCGACCTCCTTAATCATTGATAGAG |
| | for | TTATTTTACCACTCCCTATCAGTGA |
| | | TAGAGAAAA |
| 21 | RNAst-new-back | TAGCGGTGGTTTTTTTGTTGAGCTC |
| | | GGTACCCGGGGATC |
| 22 | RNAst-new-for | GGAACGGTGGTTTGTTTGCGCCTAG |
| | | TTCTCGATGGTTGAG |
| 23 | TN7-L3-GFP-back | CTTCCATTGACCGAAGTCGTCTCGC |
| | | GTCTATGGATGAAAGGAGAAGAACT |
| | | TTTCACTG |
| 24 | TN7-RBS-L2-for | CCTTTTTCTCAACCATCGAGAACTA |
| | | GGCCAATTCATATGTATATCTCCTT |
| | | CTTAAAGTTAA |

### REFERENCES

Amarzguioui, M., Prydz, H. (1998) Hammerhead ribozyme design and application. CMLS, Cell. Mol. Life Sci. 54, 1175-1202.
Balbas, P., X. Soberon, F. Bolivar, & R. L. Rodriguez (1988) The plasmid, pBR322. Biotechnol. 10, 5-41
Bhagwat, A. S., Person, S. (1981) Structure and properties of the region of homology between plasmids pMB1 and ColE1. Mol Gen Genet 182, 505-507.
Beuning, P. J., Nagan, M. C., Cramer, C. J., Musier-Forsyth, K., Josep-Lluis, G., Bashford, D. (2002) Efficient amnioacylation of the tRNA^{Ala} acceptor stem: Dependance on the 2:71 basepair. RNA 8, 659-670.
Bolivar, F. (1979) Molecular cloning vectors derived from the CoLE1 type plasmid pMB1. Life Sci. 25, 807-817
Casali, N. (2003) Escherichia coli host strains. Methods Mol. Biol. 253, 27-48.
Cesareni G, Helmer-Citterich M, Castagnoli L. (1991) Control of ColE1 plasmid replication by antisense RNA. Trends Genet., 7, 230-5.
Chan P T, Ohmori H, Tomizawa J, Lebowitz J. (1985) Nucleotide sequence and gene organization of ColE1 DNA. J. Biol. Chem. 260, 8925-35.
Chang, A. C. Y., Cohen, S. N. (1978) Construction and characterization of amplifiable multicopy DNA cloning vehicles derived from the P15A cryptic miniplasmid. Journal of Bacteriology 134, 1141-1156.
Chen, W., Graham, C., and Ciccarelli, R. B. (1997). Automated fed-batch fermentation with feed-back controls based on dissolved oxygen (DO) and pH for production of DNA vaccines. J. Ind. Microbiol. Biotechnol., 18, 43-48.
Craig, N. L: Transposon TN7. (1989) In Berg, D. E and Howe, M. H. (Eds.), Mobile DNA. American Society of Microbiology, Washington D.C., 211-225.
Datsenko K, Wanner B. (2000) One-step inactivation of chromosomal genes in Escherichia coli K-12 using PCR products. Proc. Natl. Acad. Sci. USA 97, 6640-6645.
Diaz, R., and Staudenbauer, W. L. (1982) Origin and direction of mini-R1 plasmid DNA replication in cell extracts of Escherichia coli. J Bacteriol. 150, 1077-1084
Eguchi Y, Tomizawa J. (1991a) Complexes formed by complementary RNA stem loops: Their formations, structures and interaction with ColE1 Rom protein. J Mol Biol 220:831-842.
Eguchi Y, Itoh T, Tomizawa J. (1991b) Antisense RNA. Annu Rev Biochem 60:631-52.
Fürste J, Pansegrau W, Frank R, Blöcker H, Scholz P, Bagdasarian M, Lanka E. (1986) Molecular cloning of the plasmid RP4 primase region in a multi-host-range tacP expression vector. Gene 48, 119-131.
Grabherr, R., Nilsson, E., Striedner, G. and Bayer, K. (2002) Stabilising plasmid copy number to improve recombinant protein production. Biotechnol. Bioeng. 77, 142-147.
Gregorian R, Crothers D. (1995) Determinants of RNA Hairpin Loop-Loop Complex Stability. J. Mol. Biol. 248, 968-984.
Guzman L M, Belin D, Carson M J, Beckwith J. Tight regulation, modulation, and high-level expression by vectors containing the arabinose PBAD promoter. J Bacteriol., 1995, July 177(14):4121-30.
Helinski, A Toukdarian, and R Novick, Replication control and other stable maintenance mechanisms of plasmids. In: Escherichia coli and Salmonella. Cellular and Molecular Biology. Ed: Neidhardt F, American Society for Microbiology Press, Washington, D.C., 2295-2324 (1996).
Kues, U. and Stahl, U., (1989) Replication of plasmids in gram negative bacteria. Microbiol. Rev. 53, 491-516.
Jarvis et al., 1996, Optimizing the Cell Efficacy of Synthetic Ribozymes, SITE SELECTION AND CHEMICAL MODIFICATIONS OF RIBOZYMES TARGETING THE PROTO-ONCOGENE c-myb*-. American Soc. for Biochem. and Mol. Biol., Vol. 271, No. 46, Issue of Nov. 15, 1996, pp. 29107-29112.
Lahijani, R., Hulley, G., Soriano, G., Horn, N. A., Marquet, M. (1996) High-yield production of pBR322-derived plasmids intended for human gene therapy by employing a temperature-controllable point mutation. Human Gene Therapy 7, 1971-1980.
Lin, E. C. C. (1984) Bacteria, Plasmids, and Phages. An Introduction to Molecular Biology. Cambridge, Massachusetts: Harvard University Press. p. 11-18.
Lin-Chao S, Bremer H. (1987) Activities of the RNAI and RNAII promoters of plasmid pBR322. J Bacteriol, 169(3):1217-22.
Lin-Chao S, Cohen S N. (1991) The rate of processing and degradation of antisense RNAI regulates the replication of ColE1-type plasmids in vivo. Cell 65,1233-42.
Mairhofer, J., Pfaffenzeller, I., Merz, D. and Grabherr, R. (2008) A novel antibiotic free plasmid selection system: advances in safe and efficient DNA therapy. Biotechnology Journal 3, 83-89. DOI: 10.1002/biot.200700141
Makrides SC. (1996) Strategies for achieving high-level expression of genes in Escherichia coli. Microbiol Rev. 60(3):512-38.
Merlin S, Polisky B. (1995) Assessment of quantitative models for plasmid ColE1 copy number control. J Mol Biol. 248, 211-9.
O'Kennedy, R. D., Baldwin, C., and Keshavarz-Moore, E., (2003). Effects of growth medium selection on plasmid DNA production and initial processing steps. J. Biotechnol. 76, 175-183.
Reinikainen, P., Lande, M., Karp, M., Suaminen, I., Markkanen, P., Mantsala, P. (1988) Phage lambda P(L) promoter controlled α-amylase expression in Escherichia coli during fermentation. Biotechnology Letters 10, 149-154.
Reznikoff W., (1992) The lactose operon-controlling elements: a complex paradigm. Mol Microbiol. 6(17): 2419-2422.
Rogers M, Ekaterinaki N, Nimmo E, Sherratt D. (1986) Analysis of Tn7 transposition. Mol. Gen. Genet. 205, 550-6.
Sarkar N, Cao G J, Jain C. (2002) Identification of multicopy suppressors of the pcnB plasmid copy number defect in Escherichia coli. Mol. Genet. Genomics 268,62-9.
Schleif R. (2000) Regulation of the L-arabinose operon of Escherichia coli. Trends Genet. 16(12):559-65.
Sternberg N and Hoess R. The molecular genetics of bacteriophage P1. Annu Rev Genet 1983; 17 123-54.
Striedner G, Cserjan-Puschmann M, Pötschacher F, Bayer K., 2003 Biotechnol Prog. September-October; 19(5):1427-32.
Studier F W, Moffatt B A. 1986. Use of bacteriophage T7 RNA polymerase to direct selective high-level expression of cloned genes. J Mol Biol. 189, 113-30.
Tomizawa, J., Itoh, T. (1981) Plasmid ColE1 incompatibility determined by interaction of RNAI with primer transcript. Proc Natl Acad Sci USA 78, 6096-6100.
Tomizawa, J. (1984) Control of ColE1 plasmid replication: the process of binding of RNAI to the primer transcript. Cell 38, 861-870.
Tomizawa J. (1986) Control of Plasmid Replication: Binding of RNAI to RNAII and Inhibition of Primer Formation. Cell. 47:89-97.
Tomizawa J. (1990a) Control of ColE1 plasmid replication. Intermediates in the binding of RNA I and RNA II. J Mol Biol. April 20; 212(4):683-94.
Tomizawa J. (1990b) Control of ColE1 plasmid replication. Interaction of Rom protein with an unstable complex formed by RNA I and RNA II. J Mol Biol. April 20; 212(4):695-708.
Waddell, C. S., Craig, N. L. (1988) Tn7 transposition two transposition pathways directed by five Tn7-encoded genes. Genes Dev. 2, 137-49.
Wang et al. (2001). Medium design for Plasmid DNA based on stoichimetric model. Proc. Biochem. 36, 1085-1093.
Wang, Z., Yuan, Z., Xiang L., Shao, J., Wegrzyn, G. (2006) tRNA-dependant cleavage of ColE1 plasmid encoded RNAI. Microbiology 152, 3467-3476.
Wróbel, B. and Wegrzyn, G. (1998). Replication regulation of ColE1-like plasmids in amino acid-starved Escherichia coli. Plasmid 39, 48-62.
Vieira, J. & J. Messing (1982) The pUC plasmids, an M13mp7-derived system for insertion mutagenesis and sequencing with synthetic universal primers. Gene 19, 259-268.
Yu, D., Ellis, H., Lee, C., Jenkins, N., Copeland, N., Court, D. An efficient recombination system for chromosome engineering in Escherichia coli. PNAS. 2000, May 23, Vol. 987, No. 11, 5978-5983.

1) A host-vector system comprising a non-naturally occurring bacterial host cell and a plasmid, wherein said plasmid has a ColE1-type origin of replication and wherein said bacterial host cell contains, integrated in its genome under the control of an inducible promoter, a DNA sequence encoding an RNA molecule that is able to interact with and inhibit a plasmid-transcribed RNA molecule, thereby controlling plasmid replication, wherein said genome-integrated RNA molecule is selected from
a. an RNA molecule that interacts with plasmid-transcribed RNAI, whereby, upon induction of said promoter and transcription, replication of the plasmid is upregulated;
b. an RNA molecule that interacts with plasmid-transcribed RNAII, whereby, upon induction of the promoter and transcription, replication of said plasmid is downregulated, and wherein
in the case of b), the plasmid's ColE1 origin of replication is mutated such that the function of the RNAI promoter is abolished or significantly reduced while the RNAII structure is maintained.

2) The host-vector system of claim 1, wherein said RNA molecule defined in a) is selected from wild-type or modified RNAII, tRNA that is modified in its acceptor stem, ribozyme RNA recognizing plasmid-transcribed RNAI, and anti-eutE.

3) The host-vector system of claim 1, wherein said RNA molecule defined in b) is selected from RNAI, parts of RNAI or mutants of RNAI that are directed to correspondingly mutated RNAII.

4) The host-vector system of claim 1, wherein said plasmid contains a sequence encoding a protein of interest operably associated with a prokaryotic or eukaryotic promoter.

5) A plasmid with a ColE1 origin of replication that is mutated such that the function of the RNAI promoter is abolished or significantly reduced while the RNAII structure is maintained.

6) A non-naturally occurring bacterial host cell in which a plasmid with a ColE1-type origin of replication can be replicated, wherein said bacterial host cell contains, integrated in its genome, a DNA sequence encoding an RNA molecule that has the ability to interact with RNAI or RNAII transcribed from said plasmid when such plasmid is present in the host cell, wherein transcription of said DNA sequence is under the control inducible promoter, with the proviso that said DNA sequence exclusively regulates plasmid replication without being operably linked to a functional DNA sequence on the genome.

7) A method for producing plasmid DNA or a recombinant protein of interest, wherein a host-vector system of claim 1 is cultivated and wherein, when an RNA molecule as defined in a) is used, the promoter is induced after half of the cultivation period.

8) A method for producing plasmid DNA or a recombinant protein of interest, wherein a host-vector system of claim 4 is cultivated and wherein, when an RNA molecule as defined in a) is used, the promoter is induced after half of the cultivation period.

9) A method for producing plasmid DNA or a recombinant protein of interest, wherein a host-vector system of claim 1 is cultivated and wherein, when an RNA molecule as defined in b) is used, the promoter is induced early in the cultivation process and silenced at or after half of the cultivation period.

10) The method of claim 9, wherein the promoter is silenced by using a degradable inducer that is used up at or after half of the cultivation period.

11) The method of claim 10, wherein the inducer is selected from lactose and arabinose.

12) A host-vector system comprising a non-naturally occurring bacterial host cell and a plasmid with a ColE1-type origin of replication, wherein said bacterial host cell contains, integrated in its genome
i. a DNA sequence, under the control of an inducible promoter, encoding a first RNA molecule that is able to interact with and inhibit a plasmid-transcribed RNA molecule, thereby controlling plasmid replication, wherein said first RNA molecule is selected from
a. an RNA molecule that interacts with plasmid-transcribed RNAI;
b. an RNA molecule that interacts with plasmid-transcribed RNAII;
ii. a DNA sequence encoding a protein that is lethal or toxic to said cell, and, operably associated thereto,
iii. a DNA sequence encoding a second RNA molecule that has the ability to interact with a third RNA molecule that mimics RNAI and that is transcribed from said plasmid;
and wherein said plasmid contains
i. at a locus other than the ColE1-type origin of replication, a sequence, under the control of a promoter, that encodes said third RNA molecule that mimics RNAI, and
ii. in the case of using an RNA molecule defined in b), a mutated ColE1 origin of replication such that the function of the RNAI promoter is abolished or significantly reduced;
whereby, in the presence of said plasmid, said third RNAI-mimicking molecule interacts with second RNA molecule such that expression of said toxic protein is prevented and whereby, in the absence of said plasmid, said toxic protein is expressed, and whereby, upon induction of the promoter and transcription of said first RNA molecule, replication of said plasmid is upregulated in the case of a) or downregulated in the case of b).

13) A non-naturally occurring bacterial host cell in which a plasmid with a ColE1-type origin of replication can be replicated, wherein said bacterial host cell contains, integrated in its genome, a DNA sequence encoding an RNA molecule that has the ability to interact with RNAI or RNAII transcribed from a plasmid with a ColE1-type origin, when such plasmid is present in the host cell, wherein transcription of said DNA sequence is under the control of an inducible promoter, with the proviso that said DNA sequence exclusively regulates plasmid replication without being operably linked to a functional DNA sequence on the genome.
